(19) United States
(12) Patent Application Publication
(10) Pub. No.: US 2024/0075063 A1
(43) Pub. Date: Mar. 7, 2024

(54) USE OF MAIT CELLS FOR CONTROLLING GRAFT VERSUS HOST DISEASE

(71) Applicants: INSERM (INSTITUT NATIONAL DE LA SANTÉ ET DE LA RECHERCHE MÉDICALE), Paris (FR); UNIVERSITE DE PARIS, PARIS (FR); ASSISTANCE PUBLIQUE - HÔPITAUX DE PARIS, Paris (FR)

(72) Inventors: Sophie CAILLAT-ZUCMAN, PARIS (FR); Nana TALVARD-BALLAND, Paris (FR)

(21) Appl. No.: 18/256,788

(22) PCT Filed: Dec. 9, 2021
(86) PCT No.: PCT/EP2021/085062

§ 371 Date: Jun. 9, 2023

(30) Foreign Application Priority Data
Dec. 10, 2020 (EP) 20306528.9

(51) Int. Cl. A61K 35/17; A61P 35/02

(52) U.S. Cl. CPC A61K 35/17; A61P 35/02; A61K 2035/124

(57) ABSTRACT

The inventors explored in an allogeneic situation the regulatory potential of Mucosal-Associated Invariant T cells (MAIT cells), a population of unconventional T cells that exhibit potent antibacterial activity, expressing a semi-invariant TCR which recognizes vitamin B2 derivatives of microbial origin presented by the MR1 molecule. In particular, the inventors used i) an allogenic reaction model in vitro (mixed lymphocyte reaction, MLR) and ii) murine model of xenogeneic aGvHD They first verified that human MAIT cells do not proliferate in response to allogeneic stimulation in vitro (MLR) or in vivo (immunodeficient mice) alone but require for their expansion both an inflammatory environment and TCR ligation by its ligand. In contrast, MAIT cells are able to inhibit the proliferation of allospecific LT in vitro in a dose-dependent manner. Furthermore, the adoptive transfer of MAIT cells in a mouse model of xeno-GVHD resulted in a delay in early or late GvHD development. Altogether, these data describe a new regulatory function of MAIT cells in an allogeneic context, allowing us to consider their use in cell therapy to limit GvHD.

### FIELD OF THE INVENTION

The present invention is in the field of medicine, in particular immunology.

### BACKGROUND OF THE INVENTION

MAIT cells are innate-like T cells expressing a semi-invariant TCR restricted by the monomorphic MHC class-1 Related molecule MR1, which presents microbial-derived riboflavin (vitamin B2) precursors such as 5-OP-RU (1-3). Upon TCR engagement, MAIT cells proliferate, release proinflammatory cytokines and kill target cells, supporting their role in antimicrobial defense (4-6). Most, but not all, bacteria and yeasts (but not mammals) are able to synthesize riboflavin and hence provide MR1 ligand (7). This TCR-MR1 recognition pathway therefore represents a sophisticated discriminatory mechanism to target microbial antigens while sparing the host. MAIT cells can also be activated in a TCR-independent way in response to inflammatory cytokines such as IL-12 and IL-18 (8-10). MAIT cells are predominantly localized in the liver and barrier tissues, in agreement with their expression of several chemokine receptors (4, 11). They are also abundant in the adult blood, but very few in cord blood. The inventors previously showed that the postnatal expansion of MAIT cells is a very slow process requiring at least 5-6 years to reach adult levels, and likely results from repeated encounters of a few MAIT cell clones with MR1-restricted microbial antigens (12).

The curative effect of allogeneic hematopoietic stem cell transplantation (HSCT) in hematological malignancies is based on the capacity of donor T cells to eliminate residual tumor cells (graft-versus-leukemia (GVL) effect). A serious drawback is that donor T cells may also recognize normal nonhematopoietic cells, leading to graft-versus-host disease (GVHD), which is characterized by activation, expansion and migration to target tissues of donor alloreactive T cells (13-17). In the first weeks after HSCT, the T cell compartment recovers through peripheral expansion of graft-derived T cells in response to increased levels of homeostatic cytokines and to host's allogeneic antigens. Reconstitution of a fully diversified T-cell repertoire occurs only later by resumed thymic output of newly-generated naïve T cells (18). Even under favorable conditions, it takes at least 2 months to produce naive T cells, and a plateau of thymic output is reached only after 1-2 years.

The inventors previously observed that MAIT cell recovery was delayed up to 6 years after cord blood transplantation, mimicking the long postnatal expansion period. MAIT cells were undetectable in intestinal tissues at time of acute GVHD, suggesting that they do not participate to the tissue damage mediated by donor-derived alloreactive T cells (12). Other studies have confirmed that MAIT cell numbers fail to normalize for at least a year after HSCT (19-21) and have suggested that an impaired MAIT cell number is associated with an increased risk of GVHD, although the mechanisms remain unclear (19, 21, 22). Moreover, in fully-mismatched mouse HSCT models, recipient's residual MAIT cells protect from acute intestinal GVHD through microbial-induced secretion of IL-17 and inhibition of proliferation of donor-derived alloreactive T-cells (23). Recently, transcriptomic and functional studies have revealed new tissue repair and regulatory functions of MAIT cells (24-28). Given their abundance in the liver, lungs, intestine and skin, which are classical GVHD targets, these data open the possibility of exploiting the immunomodulatory properties of MAIT cells in adoptive therapy. Indeed, because they express an MR1-restricted TCR of very limited diversity and are not selected by classical MHC-peptide complexes, MAIT cells are unlikely to cause GVHD after transfer in allogeneic recipients.

### SUMMARY OF THE INVENTION

The present invention is defined by the claims. In particular, the present invention relates to the use of MAIT cells for controlling Graft Versus Host Disease.

### DETAILED DESCRIPTION OF THE INVENTION

The inventors explored in an allogeneic situation the regulatory potential of Mucosal-Associated Invariant T cells (MATT cells), a population of unconventional T cells that exhibit potent antibacterial activity, expressing a semi-invariant TCR which recognizes vitamin B2 derivatives of microbial origin presented by the MR1 molecule. In particular, the inventors used i) an allogenic reaction model in vitro (mixed lymphocyte reaction, MLR) and ii) murine model of xenogeneic aGvHD They first verified that human MAIT cells do not proliferate in response to allogeneic stimulation in vitro (MLR) or in vivo (immunodeficient mice) alone but require for their expansion both an inflammatory environment and TCR ligation by its ligand. In contrast, MAIT cells are able to inhibit the proliferation of allospecific LT in vitro in a dose-dependent manner. Furthermore, the adoptive transfer of MATT cells in a mouse model of xeno-GVHD resulted in a delay in early or late GvHD development. Altogether, these data describe a new regulatory function of MAIT cells in an allogeneic context, allowing us to consider their use in cell therapy to control GvHD.

The first object of the present invention relates to a method of controlling Graft Versus Host Disease (GVHD) in a patient after transplantation comprising administering to the patient a therapeutically effective amount of a population of MAIT cells.

As used herein, the term “Graft Versus Host Disease” or “GVHD” refers to the pathological reaction that occurs between the grafted cells and the host-recipient. Graft-versus-host-disease (GVHD) often occurs following hematopoietic cell transplantation, and rarely after solid organ transplantation. In GVHD, donor-derived alloreactive T lymphocytes recognize the recipient's tissues as foreign. Thus, they attack and mount an inflammatory and destructive response against the recipient. GVHD has a predilection for epithelial tissues, especially skin, liver, and mucosa of the gastrointestinal tract. Transplant patients with GVHD are often treated with powerful immunosuppressant agents, thereby making them even more susceptible to opportunistic infectious agents which increase tissue damage.

As used herein, the term “controlling” includes both prevention and treatment (e.g. curative treatment).

Typically, the patient is selected from the group consisting of children, young adults, middle aged adults, and the elderly adults. In some embodiments, the patient is an elderly patient, i.e. an adult patient sixty-five years of age or older.

As used herein, the term “transplantation” and variations thereof refers to the insertion of a transplant (also called graft) into a recipient, whether the transplantation is syngeneic (where the donor and recipient are genetically identical, i.e. monozygotic twins), allogeneic (where the donor and recipient are not genoidentical but of the same species), or xenogeneic (where the donor and recipient are from different species). Thus, in a typical scenario, the host is human and the graft is an isograft, derived from a human of the same or different genetic origins. Thus, in some embodiments, the donor of the transplant is a human. The donor of the transplant can be a living donor or a deceased donor, namely a cadaveric donor. In some embodiments, the transplant is an organ, a tissue or cells.

As used herein, the term “organ” refers to a solid vascularized organ that performs a specific function or group of functions within an organism. The term organ includes, but is not limited to, heart, lung, kidney, liver, pancreas, skin, uterus, bone, cartilage, small or large bowel, bladder, brain, breast, blood vessels, esophagus, fallopian tube, gallbladder, ovaries, pancreas, prostate, placenta, spinal cord, limb including upper and lower, spleen, stomach, testes, thymus, thyroid, trachea, ureter, urethra, uterus.

As used herein, the term “tissue” refers to any type of tissue in human or animals, and includes, but is not limited to, vascular tissue, skin tissue, hepatic tissue, pancreatic tissue, neural tissue, urogenital tissue, gastrointestinal tissue, skeletal tissue including bone and cartilage, adipose tissue, connective tissue including tendons and ligaments, amniotic tissue, chorionic tissue, dura, pericardia, muscle tissue, glandular tissue, facial tissue, ophthalmic tissue. In a particular embodiment of the invention, the transplant is a cardiac allotransplant. As used herein, the term “cells” refers to a composition enriched for cells of interest, preferably a composition comprising at least 30%, preferably at least 50%, even more preferably at least 65% of said cells. In some embodiments the cells are selected from the group consisting of multipotent hematopoietic stem cells derived from bone marrow, peripheral blood, or umbilical cord blood; or pluripotent (i.e. embryonic stem cells (ES) or induced pluripotent stem cells (iPS)) or multipotent stem cell-derived differentiated cells of different cell lineages such as cardiomyocytes, beta-pancreatic cells, hepatocytes, neurons, etc. . . .

In some embodiments, the method of the present invention is particularly suitable in the context of allogeneic hematopoietic stem cell transplantation (HSCT) and thus comprises multipotent hematopoietic stem cells, usually derived from bone marrow, peripheral blood, or umbilical cord blood.

As used herein, the term “hematopoietic stem cell” or “HSC” refers to blood cells that have the capacity to self-renew and to differentiate into precursors of circulating mature blood cells. These precursor cells are immature blood cells that cannot self-renew and differentiate into circulating mature blood cells. Within the bone marrow microenvironment, the stem cells self-renew and maintain continuous production of hematopoietic cells that give rise to all mature blood cells throughout life. In some embodiments, the hematopoietic progenitor cells or hematopoietic stem cells are isolated from peripheral blood cells.

As used herein, the term “bone marrow transplantation” or “hematopoietic stem cell transplantation” used herein should be considered as interchangeable, referring to the transplantation of hematopoietic stem cells in some form to a recipient. The hematopoietic stem cells do not necessarily have to be derived from bone marrow, but could also be derived from other sources such as umbilical cord blood or mobilized PBMC. Generally, there are two types of HSCTs: autologous and allogeneic transplantation. HSCT can be curative for patients with hematopoietic cell malignancies, especially leukemia and lymphomas, but also for non-malignant hematologic diseases such as thalassemia, sickle cell disease, aplasia, metabolic diseases, severe immune deficiency . . . . However, an important limitation of allogeneic HCT is indeed the development of GVHD, which occurs in a severe form in about 30-50% of humans who receive this therapy.

In some embodiments, the patient suffers from a hematopoietic cell malignancy. Examples of hematopoietic cell malignancies that are cancers include leukemias, lymphomas and multiple myelomas. Examples of leukemias include acute lymphoblastic leukemia (ALL), acute myelogenous leukemia (AML) and chronic myelogenous leukemia (CIVIL). Examples of lymphomas include Hodgkin's disease and its subtypes; non-Hodgkin lymphomas and its subtypes including chronic lymphocytic leukemia (CLL), small lymphocytic lymphoma (SLL), diffuse large B-cell lymphoma (DLBCL), follicular lymphoma (FL), mantle cell lymphoma (MCL), hairy cell leukemia (HCL), marginal zone lymphoma (MZL), Burkitt's lymphoma (BL), Post-transplant lymphoproliferative disorder (PTLD), T-cell prolymphocytic leukemia (T-PLL), B-cell prolymphocytic leukemia (B-PLL), Waldenström's macroglobulinemia/Lymphoplasmacytic lymphoma and other natural killer cell (NK-cell) or T-cell lymphomas. Examples of other malignant conditions which are hematopoietic cell malignancies include myelodysplastic syndrome (MDS); myeloproliferative diseases such as polycythemia vera (i.e., PV, PCV or polycythemia rubra vera (PRV)), essential thrombocytosis (ET), myelofibrosis; and diseases with features of both myelodysplastic syndromes and myeloproliferative diseases such as chronic myelomonocytic leukemia (CMML), juvenile myelomonocytic leukemia (JMML), atypical chronic myeloid leukemia (aCML) and myelodysplastic/myeloproliferative disease.

In some embodiments, the patient has undergone a cytoablative therapy. As used herein, the term “cytoablative therapy” has its general meaning in the art and refers to therapy that induce cytoablative effects on rapidly-proliferating cells via several different mechanisms, ultimately leading to cell cycle arrest and/or cellular apoptosis. Typically cytoablative therapy includes chemotherapy and radiotherapy.

As used herein, the term “MAIT cells” or “Mucosal-Associated Invariant T cells” refers to a population of T cells present in mammals, preferably humans, that display a semi-invariant TCR alpha chain comprising Vα7.2-Jα33 (in humans), a CDR3 of constant length, and a limited number of Vβ segments (see, e.g., Lantz and Bendelac. 1994. J. Exp Med. 180:1097-106; Tilloy et al., J. Exp. Med., 1999, 1907-1921; Treiner et al. (2003) Nature 422:164-169, the entire disclosures of each of which are herein incorporated by reference). MAIT cells are generally CD8⁺ (expressing mostly the homodimeric form of CD8αα) or CD4⁻/CD8⁻ (DN), rarely CD4+, and are restricted by the non-classical MHC class I Related molecule MR1. For the purposes of the present invention, any T cells that express the invariant Vα7.2-Jα33 alpha TCR chain are considered to be MAIT cells. Typically, the alpha chain is associated with an invariant CDR3 and with either Vβ2 or Vβ13.

As used herein, the term “population” refers to a population of cells, wherein the majority (e.g., at least about 50%, preferably at least about 60%, more preferably at least about 70%, and even more preferably at least about 80%) of the total number of cells have the specified characteristics of the cells of interest (i.e. MAIT cells).

The population of MAIT cells can be prepared according to any method well-known in the art. Typically, said methods comprise a step of providing MAIT cells from a cell culture or from a blood sample from an individual subject or from blood bank. MAIT cells preferably derive from a donor subject, in particular a healthy donor subject. The cells can be acquired from blood samples (including peripheral blood and cord blood) as well as samples resulting from one or more processing steps, such as separation, centrifugation, genetic engineering, washing, and/or incubation. In some embodiments, the sample from the donor comprises peripheral blood mononuclear cells (PBMCs). In some embodiments, MAIT cells are collected from any location in which they reside in the subject including, but not limited to peripheral blood, peripheral blood mononuclear cells (PBMCs), bone marrow, cord blood. In some embodiments, the MAIT cells are collected by apheresis, in particular by leukapheresis.

As known to one of skill in the art, various methods are readily available for isolating immune cells from a subject or can be adapted to the present application, for example using Life Technologies Dynabeads® system; STEMcell Technologies EasySep™, RoboSep™ RosetteSep™, SepMate™; Miltenyi Biotec MACS™ cell separation kits, cell surface marker expression and other commercially available cell separation and isolation kits (e.g., ISOCELL from Pierce, Rockford, IL). MAIT cells may be isolated through the use of beads or other binding agents available in such kits specific to MAIT cell surface markers. In some embodiments, the isolation is affinity- or immunoaffinity-based separation. For example, the isolation in some aspects includes separation of MAIT cells based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner. Such separation steps can be based on positive selection, in which the MAIT cells having bound the reagents are retained for further use, and/or negative selection, in which the MAIT cells having not bound to the antibody or binding partner are retained. In some embodiments, multiple rounds of separation steps are carried out, where the positively or negatively selected fraction from one step is subjected to another separation step, such as a subsequent positive or negative selection. In some embodiments, MAIT cell population is collected and enriched via flow cytometry, in which cells stained for multiple cell surface markers are carried in a fluidic stream, such as by fluorescence-activated cell sorting (FACS), including preparative scale (FACS) and/or microelectromechanical systems (MEMS) chips, e.g., in combination with a flow-cytometric detection system. Such methods allow for positive and negative selection based on multiple markers simultaneously. In some embodiments, the isolation of MAIT cells is based on positive or high surface expression of CD3, CD8, Vα 7.2, CD161 CD26 and/or IL-18Ra (CD218a), and/or optionally on the presence of NKG2D receptor. In some embodiments, the MAIT cells are positively isolated using beads coated with an antibody, in particular an anti-Vα7.2 antibody and an anti-IL18Rα or anti-CD161 or anti-CD26 antibody. The isolated MAIT cells may be used directly, or they can be stored for a period of time, such as by freezing.

In some embodiments, the MAIT cells can be activated and/or expanded before being administered to the patient. In some embodiments, the MAIT cells are incubated in stimulatory conditions or in the presence of a stimulatory agent. Such conditions include those designed to induce proliferation, expansion, activation, and/or survival of MAIT cells, and/or to mimic antigen exposure. The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors and any other agents designed to activate the MAIT cells. For example, MAIT cells can be incubated with an anti-CD3 antibody and/or an anti-CD28 antibody under conditions stimulating proliferation of the cells. In some embodiments, the MAIT cells of the invention can be expanded in vitro by co-culturing with tissue or cells. In some embodiments, the MAIT cells are expanded by co-culturing with feeder cells, such as non-dividing PBMC. In some aspects, the non-dividing feeder cells can comprise irradiated PBMC feeder cells, in particular autologous or allogeneic irradiated PBMC, or other cells expressing MR1. In some embodiments, MAIT cells are expanded in vitro by CD3/CD28 stimulation in presence of autologous or allogeneic irradiated PBMCs and IL-2, IL7, IL-12, IL-18 and/or IL-15 cytokines.

In some embodiments, MAIT cells are expanded and/or activated in vitro in the presence of MAIT cell activating ligands such as 5-OP-RU and/or 5-OE-RU. In some embodiments, the method comprises a step of preferential in vitro MAIT cell expansion from a cell sample of a donor, in particular from PBMCs of a donor. Preferential in vitro MAIT cell expansion can be carried out by culturing PBMCs from a donor in the presence of synthetic 5-OP-RU, and optionally with cytokines. In particular, PBMCs from a donor are cultured in the presence of 5-OP-RU and IL-2 (such as rhuIL-2). In some embodiments, the MAIT cells are preferably ex vivo expanded for at least about 5 days, preferably not less than about 10 days, more preferably not less than about 15 days and most preferably not less than about 20 days before administration to the patient. In some embodiments, the MAIT cells have been expanded at least about 100 fold, preferably at least about 200 fold, and more preferably at least about 400 fold, preferably at least about 600 fold, more preferably at least about 1000 fold and even more preferably at least about 1500 fold compared to day 0 of expansion, before administration to a patient. In some embodiments, the method of preparation includes steps for freezing, e.g., cryopreserving, the cells, either before or after isolation, incubation, and/or engineering.

The population of MAIT cells prepared as described above is thus utilized in methods and compositions for adoptive immunotherapy in accordance with known techniques, or variations thereof that will be apparent to those skilled in the art based on the instant disclosure. In some embodiments, the MAIT cells are formulated by first harvesting them from their culture medium, and then washing and concentrating the cells in a medium and container system suitable for administration (a “pharmaceutically acceptable” carrier) in a treatment-effective amount. Suitable infusion medium can be any isotonic medium formulation, typically normal saline, Normosol R (Abbott) or Plasma-Lyte A (Baxter), but also 5% dextrose in water or Ringer's lactate can be utilized. The infusion medium can be supplemented with human serum albumin. A treatment-effective amount of cells in the composition is dependent on the relative representation of the MAIT cells with the desired specificity, on the age and weight of the recipient. These amount of cells can be as low as approximately 10³/kg, preferably 5×10³/kg; and as high as 10⁷/kg, preferably 10⁸/kg. The number of MAIT cells will depend upon the ultimate use for which the composition is intended, as will the type of cells included therein. For example, the population will contain greater than 70%, generally greater than 80%, 85% and 90-95% of MAIT cells. For uses provided herein, the cells are generally in a volume of a liter or less, can be 500 ml or less, even 250 ml or 100 ml or less. The clinically relevant number of immune cells can be apportioned into multiple infusions that cumulatively equal or exceed the desired total amount of cells.

The invention will be further illustrated by the following figures and examples. However, these examples and figures should not be interpreted in any way as limiting the scope of the present invention.

### FIGURES

FIG. 1: MAIT cells dose-dependently inhibit the in vitro proliferation of alloreactive T cells MAIT cells restrain alloreactive T-cell proliferation in a classic mixed lymphocyte reaction (MLR) where CFSE-labelled CD4 T cells (responding cells) are cultured with allogeneic CD3-negative antigen presenting cells (stimulating cells) in the presence of MAIT cells (or effector memory CD8 T cells as control), at different MAIT: responding cell ratios.

FIG. 2: Adoptive transfer of human MAIT cells protect from xenogeneic GVHD in immunodeficient NSG mice injected with human PBMCs. A. NSG immunodeficient mice were injected with 5×106 human total PBMCs or MAIT-depleted PBMCs. GVHD score was determined according to weight loss, posture, mobility and hair loss. Absence of MAIT cells within human PBMCs significantly increased weight loss and GVHD score. B. NSG immunodeficient mice injected with 5×10⁶ human total PBMCs with or without addition of 1×10⁶ MAIT cells at day 0, 10 or 25. In all conditions, adoptive transfer of MAIT cells significantly reduced the GVHD score (left panel) and increased mice survival (right panel).

### EXAMPLE 1

### Patients and Methods:

### Patients

Three independent cohorts of patients undergoing allogeneic HSCT for a hematological malignancy were studied.

Patient and graft characteristics are described in Table 1.

Cohort 1 included 40 children recipients, for whom peripheral blood mononuclear cells (PBMCs) samples were prospectively collected at Robert Debré Hospital between January 2013 and December 2015. Blood samples were collected prior to conditioning (±day −15 before HSCT) and at 1, 3, 6, 12 and 24 months after HSCT as the standard of care for assessment of immunologic recovery. Patients who died before day 180 were not included in the analysis.

Cohort 2 included 64 additional HSCT children in stable remission for whom PBMCs samples were collected at time of a routine visit at Robert Debré Hospital 2 to 16 years after HSCT. All children from cohorts 1 and 2 received unmanipulated bone marrow transplant from HLA-matched sibling donor (MSD) or unrelated donor (MUD). Myeloablative conditioning was provided with VP16 and total body irradiation (TBI), or with cyclophosphamide and busulfan. In vivo T-cell depletion by ATG was given in the majority of MUD recipients. Primary prophylaxis of GVHD consisted of a calcineurin inhibitor alone (MSD recipients) or with methotrexate (MUD recipients).

Cohort 3 included 49 adult donor/recipient pairs for whom frozen annotated PBMCs were provided by the CRYOSTEM consortium (https://doi.org/10.25718/cryostem-collection/2018) and SFGM-TC (Societe Francophone de Greffe de Moelle-Thérapie Cellulaire). Patients received bone marrow or peripheral blood stem cells from a matched sibling donor, and were given myeloablative or non-myeloablative conditioning. Blood samples were collected before conditioning, and at 3 and 12 months post-HSCT in the absence of aGVHD. In case of aGVHD, samples were collected at the time of diagnosis before any treatment, one month later and at 12 months.

### Cells and Reagents

PBMCs were isolated and used immediately or frozen. 5-OP-RU was synthesized as described in (29-31). Human MAIT cells were expanded for 6 days in human T-cell culture medium (RPMI-1640, Invitrogen, Life Technologies) containing 10% human AB serum (EuroBio), IL-2 (100 U/mL, Miltenyi) and 300 nM 5-OP-RU.

### Flow Cytometry

MAIT cells were analyzed on fresh whole blood, or on isolated PBMCs where indicated. Multiparametric 14-color flow cytometry analyses were performed as described in Supplementary data. MAIT cells were defined as CD3⁺CD4⁻CD161^{high}Vα7.2⁺ T cells in the first part of the study (HSCT patients). This population fully overlapped with the population labeled by MR1:5-OP-RU tetramers (31). Thereafter, we used the specific MR1:5-OP-RU tetramer when it became available (NIH tetramer core facility).

### In Vitro Stimulations

Human carboxyfluorescein succinimidyl ester (CFSE)-labeled (1 μM) PBMCs were cultured in RPMI-1640 supplemented with IL-2 (20 or 100 U/mL), IL-15 (50 ng/mL), IL-7 (10 ng/mL, all from Miltenyi), or IL-12/IL-18 (50 ng/mL each, R&D Systems) and/or 300 nM 5-OP-RU. For mixed lymphocyte reactions, CFSE-labeled PBMCs used as responders (1×10⁶/ml) were incubated with γ-irradiated allogeneic stimulator PBMCs (1:1 ratio) in 96-well round-bottom plates. Cells were harvested on day 6 and stained before flow cytometry analysis.

### Adoptive Transfer of Xenogeneic Cells

NOD-Scid-IL-2Rγ^{null} (NSG) mice (Jackson laboratory, Bar Harbor, MI) were housed under specific pathogen-free conditions in the animal facility of St-Louis Research Institute. Eight-to 10-week old female mice were used after approval of all procedures and protocols by the Institutional animal Care and Use Ethics Committee (CE121 #16624). Mice were irradiated (1.3 Gy) 24 hours prior to injection of 5×10⁶ human PBMCs in the caudal vein. Development of GVHD was monitored 3 times per week based on weight loss, hunching posture, reduced mobility and hair loss. Human chimerism in peripheral blood (percent of human CD45⁺ cells) was assessed weekly. Where indicated, mice were given 5-OP-RU (1 nmol i.p. every 3 days from the day of PBMCs infusion) and/or human IL-15 (0.1 or 0.5 μg i.p. every 3 days). Mice were sacrificed at the indicated time, or when weight loss was >15%. Peripheral blood, spleen, liver, lungs and intestine were harvested, and cells were isolated as described in Supplementary data.

### Statistics

Differences between groups were analyzed using non-parametric tests for paired (Wilcoxon) and unpaired (Mann-Whitney or Kruskall-Wallis) groups, or two-way ANOVA. Correlations were assessed using the Spearman's rank correlation. Two-sided P values <0.05 were considered significant. Analyses were performed using Prism software v.6 (GraphPad). All data including outliers were included with one pre-determined exception: flow cytometry cell-subset percentages were considered non-evaluable if the parent subset contained <100 events.

### Study Approval

The study was carried out with the approval of the Robert Debré Hospital Ethics Committee (HREC 2013/49) and the CPP Ile de France IV (2015/03NICB), in agreement with the principles of the Declaration of Helsinki and French legislation. All subjects (or their parents for the children cohort) provided written informed consent. The study was registered in a public trial registry: ClinicalTrials.gov number NCT0240.

### Results:

1/ MAIT Cell Reconstitution is Delayed for Several Years after HSCT

Our previous findings have shown that it takes up to 6 years to recover normal MAIT cell values after cord blood transplantation, suggesting that MAIT cells do not proliferate in response to allogeneic stimulation (12). However, MAIT cells in cord blood are naïve, and their number is 1-2 logs lower than in adult blood, which could contribute to this slow recovery. We therefore analyzed the kinetics of MAIT cell reconstitution in other HSCT settings.

Forty children who received an unmanipulated bone marrow transplant after myeloablative conditioning were studied longitudinally up to 24 months after HSCT. While the number of conventional T cells (Tconv) gradually increased from 1 month after transplantation and returned almost to normal after one year, there was virtually no increase in the number of MAIT cells during the study period. Two years after HSCT, MAIT cell values remained 5 times lower than in age-matched donors (data not shown). The number of Tconv and MAIT cells before and up to 3 months after HSCT was lower in matched unrelated donor (MUD) than in matched sibling donor (MSD) recipients, likely due to a longer time to transplant and more frequent use of in vivo T-cell depletion in MUD recipients. However, while Tconv reached comparable values after 3 months regardless of the donor type, MAIT cell numbers remained 2 times lower in MUD recipients than in MSD recipients (data not shown).

To extend our findings to a longer follow-up period, we performed a cross-sectional analysis of 64 additional children at time of a routine visit 2 to 16 years after transplantation. As observed after cord blood transplantation (12), the number of MAIT cells increased very slowly to reach a plateau approximately 6 years after HSCT, regardless of the donor type (data not shown). This slow recovery was not related to the underlying malignancy, gender or age of the recipient, pre-HSCT conditioning (with or without TBI) or duration of immunosuppressive treatment (data not shown). Because microbial infections have been associated with modifications of MAIT cell frequencies in the peripheral blood (5, 32-34), we considered children who presented severe microbial infection in the first 3 months after HSCT and found no impact on MAIT cell recovery (data not shown).

T-cell reconstitution is impaired in patients with aGVHD, at least in part because of defective thymic production of HSC-derived T cells (18). We previously observed that thymus derived naïve MATT cells appeared in the peripheral blood between 6 and 12 months after cord blood transplantation (12). Here we found that expansion of MAIT cells after 6 months tended to be lower in patients with severe (grade 3-4) aGVHD compared to those without or with mild (grade 1-2) aGVHD (data not shown), as also observed for Tconv cells. These data suggested that altered recovery is a consequence of aGVHD-mediated decreased thymic output of naive MAIT and Tconv cells.

To further explore the potential link between MAIT cell recovery and aGVHD, we retrospectively analyzed 49 adult HSCT donor/recipient pairs from the national CRYOSTEM consortium. As in children, MAIT cells did not expand during the study period while the number of Tconv increased sharply. One year after HSCT, MAIT cell numbers remained 4 times lower in the recipients than in their respective donors (data not shown). MAIT cell recovery was not influenced by the conditioning regimen (myeloablative or non-myeloablative) or source of HSC (peripheral blood or bone marrow) (data not shown). As observed in children, As in children, MAIT cell recovered was slightly slower in patients with severe aGVHD than in those without or with mild aGVHD (data not shown). Moreover, the proportion of Ki67+ cells at the time of aGVHD, which could represent alloreactive proliferating cells, was very low in MAIT cells compared to Tconv (data not shown).

We next aimed at evaluating the potential relationship between the number of MAIT cells in the donor and their recovery after HSCT. Although we did not always have access to the bone marrow sample to quantify MAIT cells, MAIT cell frequencies were comparable in 6 available paired bone marrow and peripheral blood samples (data not shown). Therefore, we used the number of MAIT cells in the donor's peripheral blood as a surrogate for that in the bone marrow. The number of MAIT cells in the donor was significantly related to their number in the recipient one year after HSCT (data not shown). However, there was no relationship between the number of MAIT cells in the donor and occurrence of aGVHD data not shown). Altogether, these data indicate that MAIT cell recovery is impaired for several years after HSCT regardless of factors generally associated with defective Tconv reconstitution, and suggest that the mechanisms driving expansion of Tconv and MAIT cells in this setting are different.

### 3/ MAIT Cell do not Proliferate in Response to Cytokines and Alloantigen Stimulation in the Absence of MR1 Ligand.

Graft-derived conventional T cells expand through lymphopenia-induced homeostatic proliferation and response to host's allogeneic antigens. However, not all T cells respond with the same efficiency to these proliferative cues.

We first analyzed the in vitro responsiveness of MAIT cells to homeostatic cytokines. CFSE-labeled PBMCs were treated with IL-7 or IL-15, or IL-2 as control, alone or in combination with the microbial-derived MR1 ligand, 5-OP-RU, and the proliferation of MR1-tetramer⁺ MAIT cells was monitored according to CFSE dilution (data not shown). In the absence of cytokine, MAIT cells hardly responded to 5-OP-RU alone. Most MAIT cells proliferated in response to IL-15 or IL-7 (but not to IL-2) alone, but the number of divisions was significantly higher when 5-OP-RU was added to IL-15, and to a lesser extent to IL-7. IL-12 and IL-18 are induced by chemotherapy and radiation at time of pre-transplant conditioning, and could trigger TCR-independent activation of graft-derived MAIT cells. We observed a low proliferative response of MAIT cells to IL-12/IL-18 combination, which increased in the presence 5-OP-RU but remained lower than the proliferation induced by IL-15.

We next explored the capacity of MAIT cells to respond to allogeneic cells in a mixed lymphocyte reaction (MLR). Unlike Tconv, MAIT cells barely proliferated in response to allogeneic stimulation (data not shown). However, the addition of 5-OP-RU to the MLR induced a strong proliferation of MAIT cells, suggesting that cytokines (IL-2 or other) produced by neighboring alloreactive T cells during the culture period allowed MAIT cells to proliferate in response to the MR1 ligand (data not shown).

Overall, these results suggest that signals provided by allogeneic cells and (homeostatic or inflammatory) cytokines produced in the post-HSCT period are not sufficient to induce sustained expansion of MAIT cells,

### 4/ Human MAIT Cells do not Expand in Immunodeficient Mice and do not Cause Xenogeneic GVHD (Xeno-GVHD)

To further explore the potential of human MAIT cells to expand and participate to GVHD tissue lesions in vivo, we used a model of xenogeneic GVHD in which low doses of human PBMCs (huPBMCs) are injected into irradiated immunodeficient NSG mice. In this model, human T cells consistently expand in the mouse and mediate an acute GVHD-like syndrome with extensive T-cell tissue infiltration and damage of mouse skin, liver, intestine and lungs, resulting in death by 30-50 days.

Mice were injected with 5×10⁶ huPBMCs, among which MAIT cells represented around 3% of T cells. The presence of CD45+ huPBMCs was determined at different times after transfer in tissues of recipient mice, including those where MAIT cells are known to preferentially reside.

Four weeks after transfer, a variable proportion of CD45⁺ cells were found in peripheral blood and tissues, almost all of which were Tconv but less than 0.05% were MAIT cells (data not shown).

Next, mice injected with huPBMCs were monitored to evaluate aGVHD progression and euthanized when weight loss was >15% (±45 days after transfer). While a massive accumulation of Tconv was observed in particular in the spleen, lungs and liver, MAIT cells were barely detectable in all compartments (data not shown).

Human MAIT cells efficiently recognize the murine MR1 molecule (32), ruling out a defective presentation of MR1 ligands to human MAIT cells in NSG mice. However, since MAIT cells do not proliferate significantly in vitro in the absence of 5-OP-RU, the availability of MR1 ligand could be a factor limiting their expansion or survival in mice raised under specific-pathogen-free conditions. Mice were thus given 1 nmol 5-OP-RU intraperitoneally every 3 days from the day of huPBMC injection, a dose previously shown to activate endogenous MAIT cells (35). This did not result in any increase in MAIT cells in peripheral blood or tissues (data not shown).

It is not clear whether mouse cytokines can sustain homeostatic proliferation and survival of human MAIT cells in NSG mice due to a species barrier between human lymphoid cells and recipient microenvironment (36-38). This is a key question for IL-15, as it is mostly mouse-derived in the xeno-GVHD model given the low human myeloid chimerism. Indeed, we found that human MAIT cells cultured with mouse IL-15 did not proliferate at all in vitro. Proliferation was partially restored when 5-OP-RU was added to mouse IL-15, although it remained lower than with human IL-15. Murine and human IL-7 had similar effects on MAIT cell proliferation, regardless of the presence of 5-OP-RU (data not shown). Thus, IL-15 availability may be suboptimal for the proliferation of MAIT cells in NSG mice.

We therefore sought to determine the rate of division of MAIT cells after transfer into NSG mice. CFSE-labeled huPBMCs were recovered from the spleen and liver 1 week after transfer. More than 60% of Tconv had low CFSE fluorescence due to cell division. By contrast, the vast majority of MAIT cells remained CFSE^{high}, indicating that they were able to migrate to and survive in the spleen and liver, but did not divide significantly (data not shown). In parallel experiments, mice were given human IL-15, IL-7 or IL-2 plus 5-OP-RU every other day from the day of transfer. IL-7 and IL-2 weakly promoted MAIT cell division and did not modify Tconv proliferation. Conversely, IL-15 greatly enhanced the division of MAIT cells (data not shown). These results confirm that human MAIT cells do not proliferate in the absence of combined TCR and cytokine signals, consistent with in vitro observations.

We therefore treated NSG mice with human IL-15 three times per week from the day of huPBMC transfer and assessed the progression of aGVHD. Mice developed signs of severe aGVHD earlier than in the absence of IL-15 and had to be euthanized 25 day after transfer. A massive T-cell infiltration was observed in all compartments. However, MAIT cells still remained barely detectable (data not shown).

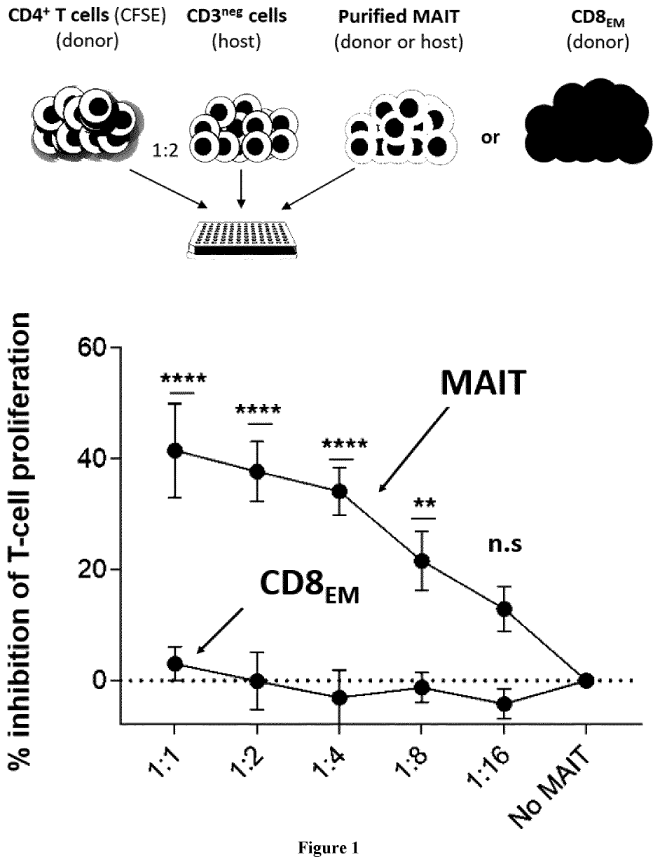

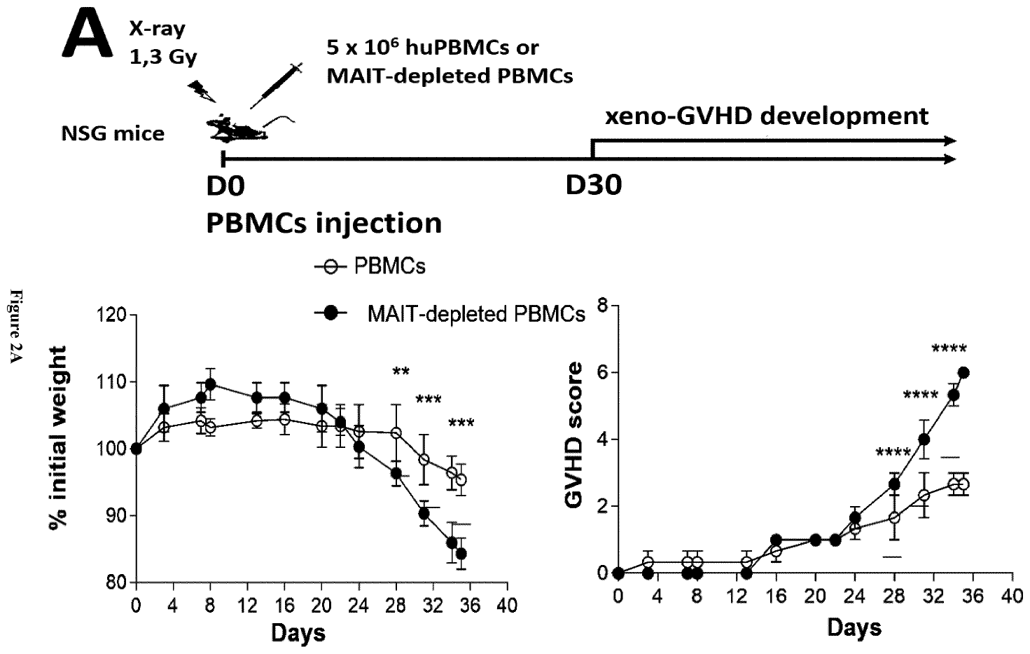

We demonstrated that MAIT cells dose-dependently inhibit the in vitro proliferation of alloreactive T cells (FIG. 1).

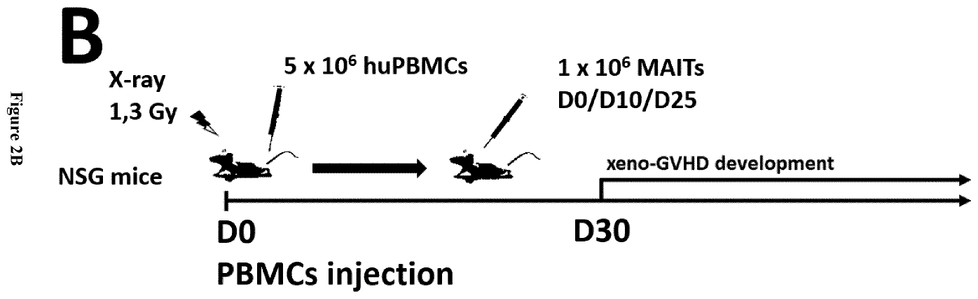

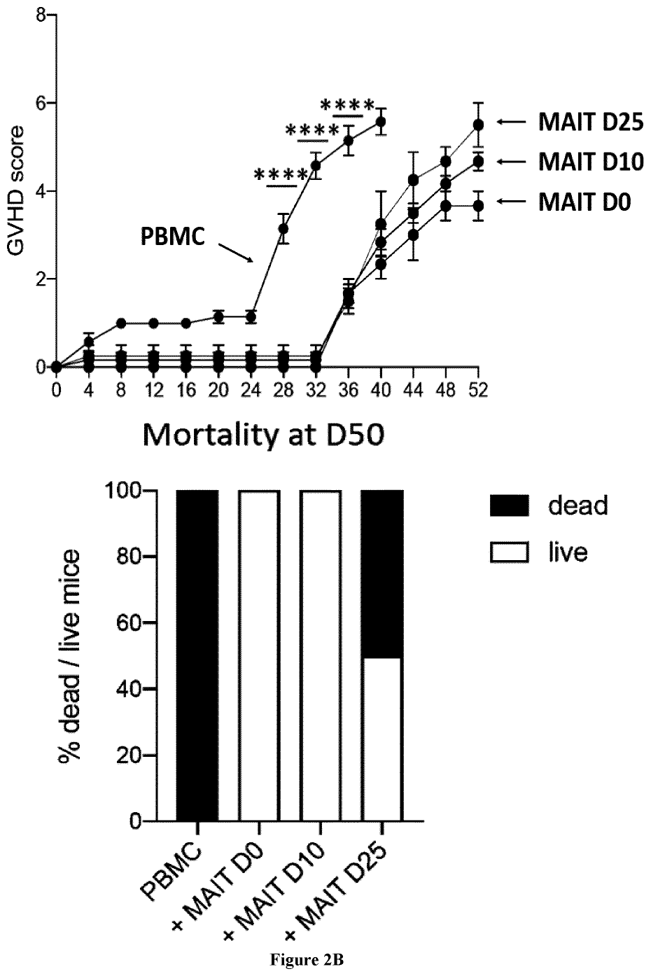

We also showed that adoptive transfer of human MAIT cells protect from xenogeneic GVHD in immunodeficient NSG mice injected with human PBMCs (FIGS. 2A and 2B).

Altogether, these results indicate that human MAIT cells, although able to survive in immunodeficient mice, do not expand nor accumulate in tissues and do no not participate to T cell-mediated xeno-GVHD.

### Discussion

The newly described immunoregulatory and tissue repair functions of MAIT cells open fascinating perspectives for their use in adoptive therapy to control immune-mediated damage in tissues where these cells are known to accumulate. However, this strategy can only be considered if MAIT cells are devoid of alloreactive potential that could lead to a GVH effect in unrelated recipients. As anticipated by the very limited diversity of their TCR that recognizes microbial antigens presented by the highly conserved MR1 molecule, here we provide evidence that MAIT cells do not respond to allogeneic signals.

Human MAIT cells are very few at birth and accumulate gradually during infancy, with an expansion of about 30 times to reach a plateau around 6 years of age (12). Several pieces of evidence suggest that the drivers of this peripheral expansion are related to successive encounters with microbes leading to an accumulation of MAIT cell clonotypes that will constitute the future MAIT cell pool (39). Indeed, MAIT cells are absent in germ-free mice and are very few in laboratory mice, but dramatically expand following challenge with riboflavin-producing microbes (32, 35, 40). Moreover, their development in mice depends on early-life exposure to defined microbes that synthesize riboflavin-derived antigens (27). In human subjects with controlled infection, MAIT cells show evidence of expansion of select MAIT cell clonotypes (41). However, only a thorough post-natal longitudinal analysis of MAIT cell levels in relation to microbial environments would be able to precisely characterize how the history of microbial infections contributes to their time-dependent expansion.

Here, we show that the reconstitution of MAIT cells after HSCT occurs over a period of at least 6 years, thus recapitulating their physiological expansion in the infancy. This reconstitution is independent of recipient- or donor-related factors such as age, underlying disease, donor type, stem cell source or conditioning regimen, but appears to be lower in patients undergoing severe aGVHD. Whether aGVHD is the cause or the consequence of this low recovery remains unclear. GVHD-induced thymic injury leads to loss of the large double-positive (DP) thymocyte population as a consequence of increased apoptotic cell death (42). This may decrease the thymic output of naïve MAIT cells, which undergo positive selection by recognizing 1 at the surface of DP thymocytes (43). In addition, loss of diversity and increased bacterial domination early after HSCT, in particular by Enterococcus, has been associated with increased risk of aGVHD, an effect dependent on the presence of T cells (44-46). One might speculate that blooming of Enterococcus (a strain unable to synthesize riboflavin) could prevent from early expansion of donor-derived MAITs due to lack of MR1-ligands. Investigating the gut metagenome or metatranscriptome of HSCT recipients for the presence of riboflavin biosynthesis genes should answer this question.

In mouse HSCT models, conditioning-resistant host residual MAIT cells promote gastrointestinal tract integrity and inhibit the proliferation of donor-derived alloreactive T-cells, thus preventing activation of alloreactive donor T cells (23). Although these findings are not directly translatable to human aGVHD due to specificities of HSCT models in mice and differences between mouse and human MAIT cells (23, 47, 48), they support our observations ant those of other groups showing an association between low MAIT cell numbers early after HSCT and aGVHD (19, 21). Altogether, these data indicate that the effect of MAIT cells after HSCT, if any, is to protect from rather than contribute to aGVHD.

Due to abundance of MAIT cells in tissues and ubiquitous expression of MR1, MAIT cell functions need to be tightly regulated. Using a classical in vitro model of alloreactivity, we show that MAIT cells do not proliferate in response to allogeneic cells, except if the TCR is engaged by MR1-ligand in the presence of soluble factors produced by alloreactive Tconv cells. Moreover, MAIT cells do not participate in the development of xeno-GVHD in NSG mice infused with huPBMCs. It is likely that the xeno-GVHD is caused by a fraction of T cells having a low frequency in donor PBMCs, which subsequently expand in mouse organs upon recognition of murine MHC (49). One cannot exclude that the NSG host with an ablated immune compartment may be less likely to provide conditions for presentation of MR1-ligands to MAIT cells or the delivery of costimulatory signals. However, MR1 is highly conserved across various species, with 90% of sequence similarity between mice and humans, so that murine MR1 can present 5-OP-RU to human MAIT cells as efficiently as human MR1 (32). That donor T cells may outcompete MAIT cells by limiting the availability of IL-15 seems unlikely, as demonstrated by the failure of exogenous human IL-15 to increase MAIT cell numbers in NSG recipients, even in the presence of MR1 ligand. Whether MAIT cells fail to traffic or find their niche in the host due to species barriers between chemokine receptors and their ligands is also unlikely given the variety of tissue homing molecules expressed on MAIT cells (4, 50).

In conclusion, MAIT cells do not expand nor accumulate in tissues in response to allogeneic stimulation. Cytokines may provide early but limited proliferation signals to graft-derived MAIT cells, at least ensuring their survival. However, sustained expansion of mature and thymus-derived MAIT cells will only occur when MR1-ligands are present together with inflammatory signals. Such restricted conditions are likely to be crucial in controlling the balance between healthy and pathological processes. These data pave the way for harnessing novel MAIT cell immunoregulatory functions in the allogeneic setting.

### Tables

TABLE 1
Characteristics of the children HSCT recipients

| | Sibling donor | Unrelated donor |
|---|---|---|
| Donor origin | 19 (47.5%) | 21 (52.5%) |
| Female gender | 6 (31.6%) | 10 (47.7%) |
| Median age of recipient, | 11 (0.67-16) | 10.5 (2-15) |
| yrs (range) | | |
| Donor age, yrs | 14 (1-21) | >18 |
| Hematological disease: | | |
| ALL | 14 (73.7%) | 9 (42.9) |
| AML | 3 (15.8%) | 8 (38%) |
| JMML | 1 (5.3%) | 2 (9.5%) |
| Lymphoma | 0 | 1 (4.8%) |
| Myelodysplasia | 1 (5.25%) | 0 |
| CML | 0 | 1 (4.8%) |
| Myeloablative | 19 (100%) | 21 (100%) |
| conditioning | | |
| In vivo T-cell | 0 | 12 (57%) |
| depletion (ATG) | | |
| Acute GVHD | | |
| Stage 0-1 | 6 (32%) | 8 (38%) |
| Stage 2/3/4 | 13 X/X/X?(68%) | 13 (62%) |

ALL: acute lymphoblastic leukemia;
AML: acute myeloid leukemia;
CML: chronic myeloid leukemia;
CLL: chronic lymphoblastic leukemia;
JMML: juvenile myelomonocytic leukemia

TABLE 2
Characteristics of the adult HSCT recipients
from the CRYOSTEM biobank

| | | Non- |
|---|---|---|
| | Myeloablative | myeloablative |
| | conditioning | conditioning |
| Pre-transplant conditioning | 24 (49%) | 25 (51%) |
| Female gender | 8 (33.3%) | 10 (40%) |
| Median age of recipient, yrs (range) | 45 (17-56) | 45 (23-65) |
| Hematological disease: | | |
| AML | 8 (33.3%) | 11 (44%) |
| ALL | 6 (25%) | 0 |
| Hodgkin lymphoma | 2 (8.3%) | 4 (16%) |
| Non Hodgkin lymphoma | 4 (16.6%) | 3 (12%) |
| MDS or MPN | 1 (4.2%) | 4 (16%) |
| Multiple myeloma | 2 (8.3%) | 1 (4%) |
| Secondary acute leukemia | 1 (4.2%) | 0 |
| CLL | 0 | 1 (4%) |
| CML | 0 | 1 (4%) |
| Stem cell source: | | |
| Peripheral blood | 8 (33.3%) | 22 (88%) |
| Bone marrow | 16 (66.7%) | 3 (12%) |
| Acute GVHD | | |
| Stage 0-1 | 16 (66.7%) | 17 (68%) |
| Stage 2-4 | 8 (33.3%) | 8 (32%) |
| Mean time from transplant to | | |
| GVHD, days (range) | 34 (16-102) | 30 (10-51) |
| In vivo T-cell depletion (ATG) | 5 (20.8%) | 13 (52%) |

ALL: acute lymphoblastic leukemia;
AML: acute myeloid leukemia;
CML: chronic myeloid leukemia;
CLL: chronic lymphoblastic leukemia;
MDS: Myelodysplastic syndrome;
MPN: myeloproliferative neoplasm

### REFERENCES

Throughout this application, various references describe the state of the art to which this invention pertains. The disclosures of these references are hereby incorporated by reference into the present disclosure.
1. Treiner E, Duban L, Bahram S, Radosavljevic M, Wanner V, Tilloy F, et al. Selection of evolutionarily conserved mucosal-associated invariant T cells by MR1. Nature. 2003; 422(6928): 164-9.
2. Kjer-Nielsen L, Patel O, Corbett A J, Le Nours J, Meehan B, Liu L, et al. MR1 presents microbial vitamin B metabolites to MAIT cells. Nature. 2012; 491(7426):717-23.
3. Corbett A J, Eckle S B, Birkinshaw R W, Liu L, Patel O, Mahony J, et al. T-cell activation by transitory neo-antigens derived from distinct microbial pathways. Nature. 2014; 509(7500):361-5.
4. Dusseaux M, Martin E, Serriari N, Peguillet I, Premel V, Louis D, et al. Human MAIT cells are xenobiotic-resistant, tissue-targeted, CD161hi IL-17-secreting T cells. Blood. 2011; 117(4):1250-9.
5. Gold M C, Cerri S, Smyk-Pearson S, Cansler M E, Vogt T M, Delepine J, et al. Human mucosal associated invariant T cells detect bacterially infected cells. PLoS Biol. 2010; 8(6):e1000407.
6. Le Bourhis L, Dusseaux M, Bohineust A, Bessoles S, Martin E, Premel V, et al. MAIT cells detect and efficiently lyse bacterially-infected epithelial cells. PLoS Pathog. 2013; 9(10): e1003681.
7. Mondot S, Boudinot P, Lantz O. MAIT, MR1, microbes and riboflavin: a paradigm for the co-evolution of invariant TCRs and restricting MHCI-like molecules? Immunogenetics. 2016; 68(8):537-48.
8. Loh L, Wang Z, Sant S, Koutsakos M, Jegaskanda S, Corbett A J, et al. Human mucosal-associated invariant T cells contribute to antiviral influenza immunity via IL-18-dependent activation. Proc Natl Acad Sci USA. 2016; 113(36):10133-8.
9. Ussher J E, Bilton M, Attwod E, Shadwell J, Richardson R, de Lara C, et al. CD161++CD8+ T cells, including the MAIT cell subset, are specifically activated by IL-12+IL-18 in a TCR-independent manner. Eur J Immunol. 2014; 44(1):195-203.
10. Wilgenburg B V, Loh L, Chen Z, Pediongco T J, Wang H, Shi M, et al. MAIT cells contribute to protection against lethal influenza infection in vivo. Nat Commun. 2018; 9(1):4706.
11. Martin E, Treiner E, Duban L, Guerri L, Laude H, Toly C, et al. Stepwise development of MAIT cells in mouse and human. PLoS Biol. 2009; 7(3):e54.
12. Ben Youssef G, Tourret M, Salou M, Ghazarian L, Houdouin V, Mondot S, et al. Ontogeny of human mucosal-associated invariant T cells and related T cell subsets. J Exp Med. 2018; 215(2):459-79.
13. Zeiser R, Blazar B R. Acute Graft-versus-Host Disease—Biologic Process, Prevention, and Therapy. N Engl J Med. 2017; 377(22):2167-79.
14. Blazar B R, Hill G R, Murphy W J. Dissecting the biology of allogeneic HSCT to enhance the GvT effect whilst minimizing GvHD. Nat Rev Clin Oncol. 2020.
15. Magenau J, Runaas L, Reddy P. Advances in understanding the pathogenesis of graft-versus-host disease. Br J Haematol. 2016; 173(2):190-205.
16. Zeiser R, Socie G, Blazar B R. Pathogenesis of acute graft-versus-host disease: from intestinal microbiota alterations to donor T cell activation. Br J Haematol. 2016; 175(2):191-207.
17. Socie G, Ritz J. Current issues in chronic graft-versus-host disease. Blood. 2014; 124(3):374-84.
18. Chaudhry M S, Velardi E, Malard F, van den Brink M R. Immune Reconstitution after Allogeneic Hematopoietic Stem Cell Transplantation: Time To T Up the Thymus. J Immunol. 2017; 198(1):40-6.
19. Bhattacharyya A, Hanafi L A, Sheih A, Golob J L, Srinivasan S, Boeckh M J, et al. Graft-Derived Reconstitution of Mucosal-Associated Invariant T Cells after Allogeneic Hematopoietic Cell Transplantation. Biol Blood Marrow Transplant. 2018; 24(2):242-51.
20. Solders M, Erkers T, Gorchs L, Poiret T, Remberger M, Magalhaes I, et al. Mucosal-Associated Invariant T Cells Display a Poor Reconstitution and Altered Phenotype after Allogeneic Hematopoietic Stem Cell Transplantation. Front Immunol. 2017; 8:1861.
21. Kawaguchi K, Umeda K, Hiejima E, Iwai A, Mikami M, Nodomi S, et al. Influence of post-transplant mucosal-associated invariant T cell recovery on the development of acute graft-versus-host disease in allogeneic bone marrow transplantation. Int J Hematol. 2018; 108(1):66-75.
22. Konuma T, Kohara C, Watanabe E, Takahashi S, Ozawa G, Suzuki K, et al. Reconstitution of Circulating Mucosal-Associated Invariant T Cells after Allogeneic Hematopoietic Cell Transplantation: Its Association with the Riboflavin Synthetic Pathway of Gut Microbiota in Cord Blood Transplant Recipients. J Immunol. 2020; 204(6):1462-73.
23. Varelias A, Bunting M D, Ormerod K L, Koyama M, Olver S D, Straube J, et al. Recipient mucosal-associated invariant T cells control GVHD within the colon. J Clin Invest. 2018; 128(5):1919-36.
24. Leng T, Akther H D, Hackstein C P, Powell K, King T, Friedrich M, et al. TCR and Inflammatory Signals Tune Human MAIT Cells to Exert Specific Tissue Repair and Effector Functions. Cell Rep. 2019; 28(12):3077-91 e5.
25. Lamichhane R, Schneider M, de la Harpe S M, Harrop T W R, Hannaway R F, Dearden P K, et al. TCR- or Cytokine-Activated CD8(+) Mucosal-Associated Invariant T Cells Are Rapid Polyfunctional Effectors That Can Coordinate Immune Responses. Cell Rep. 2019; 28(12):3061-76 e5.
26. Hinks T S C, Marchi E, Jabeen M, Olshansky M, Kurioka A, Pediongco T J, et al. Activation and In Vivo Evolution of the MAIT Cell Transcriptome in Mice and Humans Reveals Tissue Repair Functionality. Cell Rep. 2019; 28(12):3249-62 e5.
27. Constantinides M G, Link V M, Tamoutounour S, Wong A C, Perez-Chaparro P J, Han S J, et al. MAIT cells are imprinted by the microbiota in early life and promote tissue repair. Science. 2019; 366(6464).
28. Salou M, Lantz O. A TCR-Dependent Tissue Repair Potential of MAIT Cells. Trends Immunol. 2019; 40(11):975-7.
29. Soudais C, Samassa F, Sarkis M, Le Bourhis L, Bessoles S, Blanot D, et al. In Vitro and In Vivo Analysis of the Gram-Negative Bacteria-Derived Riboflavin Precursor Derivatives Activating Mouse MAIT Cells. J Immunol. 2015; 194(10):4641-9.
30. Mak J Y, Xu W, Reid R C, Corbett A J, Meehan B S, Wang H, et al. Stabilizing short-lived Schiff base derivatives of 5-aminouracils that activate mucosal-associated invariant T cells. Nat Commun. 2017; 8:14599.
31. Reantragoon R, Corbett A J, Sakala I G, Gherardin N A, Furness J B, Chen Z, et al. Antigen-loaded MR1 tetramers define T cell receptor heterogeneity in mucosal-associated invariant T cells. J Exp Med. 2013; 210(11):2305-20.
32. Le Bourhis L, Martin E, Peguillet I, Guihot A, Froux N, Core M, et al. Antimicrobial activity of mucosal-associated invariant T cells. Nat Immunol. 2010; 11(8):701-8.
33. Grimaldi D, Le Bourhis L, Sauneuf B, Dechartres A, Rousseau C, Ouaaz F, et al. Specific MAIT cell behaviour among innate-like T lymphocytes in critically ill patients with severe infections. Intensive Care Med. 2014; 40(2):192-201.
34. Ghazarian L, Caillat-Zucman S, Houdouin V. Mucosal-Associated Invariant T Cell Interactions with Commensal and Pathogenic Bacteria: Potential Role in Antimicrobial Immunity in the Child. Front Immunol. 2017; 8:1837.
35. Legoux F, Bellet D, Daviaud C, El Mon Y, Darbois A, Niort K, et al. Microbial metabolites control the thymic development of mucosal-associated invariant T cells. Science. 2019; 366(6464):494-9.
36. Roychowdhury S, Blaser B W, Freud A G, Katz K, Bhatt D, Ferketich A K, et al. IL-15 but not IL-2 rapidly induces lethal xenogeneic graft-versus-host disease. Blood. 2005; 106(7):2433-5.
37. Huntington N D, Legrand N, Alves N L, Jaron B, Weijer K, Plet A, et al. IL-15 trans-presentation promotes human N K cell development and differentiation in vivo. J Exp Med. 2009; 206(1):25-34.
38. Herndler-Brandstetter D, Shan L, Yao Y, Stecher C, Plajer V, Lietzenmayer M, et al. Humanized mouse model supports development, function, and tissue residency of human natural killer cells. Proc Natl Acad Sci USA. 2017; 114(45):E9626-E34.
39. Lantz O, Legoux F. MAIT cells: an historical and evolutionary perspective. Immunol Cell Biol. 2018; 96(6):564-72.
40. Godfrey D I, Koay H F, McCluskey J, Gherardin N A. The biology and functional importance of MAIT cells. Nat Immunol. 2019; 20(9):1110-28.
41. Howson L J, Napolitani G, Shepherd D, Ghadbane H, Kurupati P, Preciado-Llanes L, et al. MAIT cell clonal expansion and TCR repertoire shaping in human volunteers challenged with Salmonella Paratyphi A. Nat Commun. 2018; 9(1):253.
42. Krenger W, Blazar B R, Hollander G A. Thymic T-cell development in allogeneic stem cell transplantation. Blood. 2011; 117(25):6768-76.
43. Legoux F, Salou M, Lantz 0. MAIT Cell Development and Functions: the Microbial Connection. Immunity. 2020; 53(4):710-23.
44. Taur Y, Jenq R R, Perales M A, Littmann E R, Morjaria S, Ling L, et al. The effects of intestinal tract bacterial diversity on mortality following allogeneic hematopoietic stem cell transplantation. Blood. 2014; 124(7):1174-82.
45. Jenq R R, Taur Y, Devlin S M, Ponce D M, Goldberg J D, Ahr K F, et al. Intestinal Blautia Is Associated with Reduced Death from Graft-versus-Host Disease. Biol Blood Marrow Transplant. 2015; 21(8):1373-83.
46. Stein-Thoeringer C K, Nichols K B, Lazrak A, Docampo M D, Slingerland A E, Slingerland J B, et al. Lactose drives Enterococcus expansion to promote graft-versus-host disease. Science. 2019; 366(6469):1143-9.
47. Cui Y, Franciszkiewicz K, Mburu Y K, Mondot S, Le Bourhis L, Premel V, et al. Mucosal-associated invariant T cell-rich congenic mouse strain allows functional evaluation. J Clin Invest. 2015; 125(11):4171-85.
48. Rahimpour A, Koay H F, Enders A, Clanchy R, Eckle S B, Meehan B, et al. Identification of phenotypically and functionally heterogeneous mouse mucosal-associated invariant T cells using MR1 tetramers. J Exp Med. 2015; 212(7):1095-108.
49. Ehx G, Somja J, Warnatz H J, Ritacco C, Hannon M, Delens L, et al. Xenogeneic Graft-Versus-Host Disease in Humanized NSG and NSG-HLA-A2/HHD Mice. Front Immunol. 2018; 9:1943.
50. Walker L J, Kang Y H, Smith M O, Tharmalingham H, Ramamurthy N, Fleming V M, et al. Human MAIT and CD8alphaalpha cells develop from a pool of type-17 precommitted CD8+ T cells. Blood. 2012; 119(2):422-33.

1. A method of controlling Graft Versus Host Disease (GVHD) in a patient after transplantation comprising administering to the patient a therapeutically effective amount of a population of MAIT cells.

2. The method of claim 1 wherein the transplantation is an allogeneic hematopoietic stem cell transplantation (HSCT).

3. The method of claim 2 wherein the HSCT is carried out for the treatment of hematopoietic cell malignancies or non-malignant hematologic diseases.

4. The method of claim 3 wherein the subject suffers from a hematopoietic cell malignancy selected from the group consisting of leukemias, lymphomas and multiple myelomas.

5. The method of claim 2 wherein the patient has undergone a cytoablative therapy.

6. The method of claim 1 wherein the population of MAIT cells is prepared from a cell culture or from a blood sample from an individual subject or from a blood bank.

7. The method of claim 1 wherein the MAIT cells are activated and/or expanded before being administered to the patient.

8. The method of claim 3, wherein the hematopoietic cell malignancy or non-malignant hematologic disease is thalassemia, sickle cell disease, aplasia, metabolic diseases or severe immune deficiency.
